(19) United States
(12) Patent Application Publication
(10) Pub. No.: US 2022/0134071 A1
(43) Pub. Date: May 5, 2022

(54) SKIN-CARE PRODUCT AND DISPOSABLE UNDERWEAR HAVING THE SAME

(71) Applicant: Tantuc Asia LTD., Taipei City (TW)

(72) Inventor: YUN-XIAO CUI, New Taipei City (TW)

(73) Assignee: Tantuc Asia LTD., Taipei City (TW)

(21) Appl. No.: 17/468,374

(22) Filed: Sep. 7, 2021

(30) Foreign Application Priority Data
Oct. 30, 2020 (TW) 109137815

(51) Int. Cl. A61M 35/00; A61K 31/728; A41B 9/00; A41D 31/102

(52) U.S. Cl. CPC A61M 35/10; A41D 31/102; A41B 9/001; A61K 31/728

(57) ABSTRACT

A skin-care product includes a carrier, a middle pad, a protective film. The carrier has an attached surface and an engaging surface opposite to the attached surface. The attached surface is attached to an inner surface of an underwear. The middle pad is disposed on the engaging surface of the carrier and includes a maintenance ingredient for nourishing a human skin and has a contact surface. The protective film is engaged with the middle pad in a way that the protective film entirely covers the contact surface of the middle pad. The attached surface could be detachably connected to or fixed to the inner surface of the underwear. The protective film is removed before using the skin-care product, so that the contact surface of the middle pad could contact the human skin allow the user to do other housework or other activities while performing skincare, enhancing convenience.

### BACKGROUND OF THE INVENTION

### Technical Field

The present invention relates generally to maintenance supplies, and more particularly to a skin-care product and a disposable underwear with the skin-care product.

### Description of Related Art

Modern people pay more and more attention to appearance in recent years, different kinds of skincare products have emerged. However, some maintenance products that are directly applied to human skin usually restrict the user's movement. For example, when applying a facial mask, the user often adopts a lying position to facilitate the maintenance liquid in the mask to enter the facial skin; or, when applying maintenance oil on the skin of the legs and feet, the user usually sits and wait for the maintenance oil to be absorbed and dry a little before walking around to prevent the maintenance oil from contaminating items or soiling the ground. The aforementioned situations have made problems for some people who love beauty. What's more, the maintenance of some joints or private parts not only causes troubles in use, but also wastes time due to restrictions on movement during maintenance, which is inconvenient for busy people.

Taiwan invention patent 1242415 discloses a kind of maintenance shorts, which has a maintenance liquid inside to nourish the skin of the crotch, the groin, and the inner thigh, and is provided with an isolation layer on the outside to prevent the maintenance liquid from leaking out, which seems to have achieved the purpose of convenience for users. However, before the maintenance shorts being used, the maintenance liquid is likely to overflow or evaporate from the inner surface of the maintenance shorts, which may cause only little maintenance liquid left when using the maintenance shorts, so it is necessary to solve the problem.

### BRIEF SUMMARY OF THE INVENTION

In view of the above, the primary objective of the present invention is to provide a skin-care product and a disposable underwear with the skin-care product, which could facilitate users to maintain skin.

The present invention provides a skin-care product adapted to be detachably attached to an inner surface of an underwear, including a carrier, a middle pad, and a protective film. The carrier has an attached surface and an engaging surface which face opposite directions, wherein the attached surface is adapted to be attached to the inner surface of the underwear. The middle pad is disposed on the engaging surface of the carrier and comprises a maintenance ingredient for nourishing a human skin and has a contact surface. The protective film is detachably engaged with the middle pad in a way that the protective film entirely covers the contact surface of the middle pad.

With the aforementioned design, when the skin-care product is attached to the inner surface of the underwear, the maintenance of the private parts could be performed by wearing the underwear. In addition, the carrier is made of a waterproof material, thereby preventing the maintenance solution in the middle pad from leaking from a gap of the underwear to stick or drip, making it more convenient and comfortable to use. When the maintenance is completed after a predetermined time, the skin-care product could be taken off from the underwear to discard. Moreover, the protective film could prevent the maintenance solution from leaking, evaporating, or spilling before using the skin-care product.

The present invention further provides a disposable underwear with a skin-care product, including a close-fitting fabric and a skin-care product, wherein the skin-care product includes a middle pad and a protective film. The middle pad includes a maintenance ingredient for nourishing a human skin and has a first surface and a second surface which face opposite directions. The first surface is attached to an inner surface of the close-fitting fabric. The protective film is detachably attached to the second surface and entirely covers the second surface.

With the aforementioned design, the user could do other housework or other activities while performing skincare by wearing the underwear. In addition, at least a part of the disposable underwear is made of a waterproof material, thereby preventing the maintenance solution in the middle pad from leaking from a gap of the underwear to stick or drip, making it more convenient and comfortable to use. When the skin properly absorbs the maintenance ingredients after a predetermined time, the underwear could be taken off and discarded. Moreover, the protective film could prevent the maintenance solution from leaking, evaporating, or spilling before using the skin-care product.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a partial sectional schematic view of the skin-care product according to a first embodiment of the present invention;

FIG. 2 is a perspective view of the disposable skin-care bra according to a second embodiment of the present invention;

FIG. 3 is a schematic view of the disposable skin-care bra according to the second embodiment of the present invention, showing the disposable skin-care bra is in a state of use;

FIG. 4 is a right-side schematic view of FIG. 3;

FIG. 5 is a perspective view of the disposable skin-care underpants according to a third embodiment of the present invention;

FIG. 6 is a schematic view of the disposable skin-care underpants according to the third embodiment of the present invention, showing the disposable skin-care underpants is in a state of use; and

FIG. 7 is a right-side schematic view of FIG. 6.

### DETAILED DESCRIPTION OF THE INVENTION

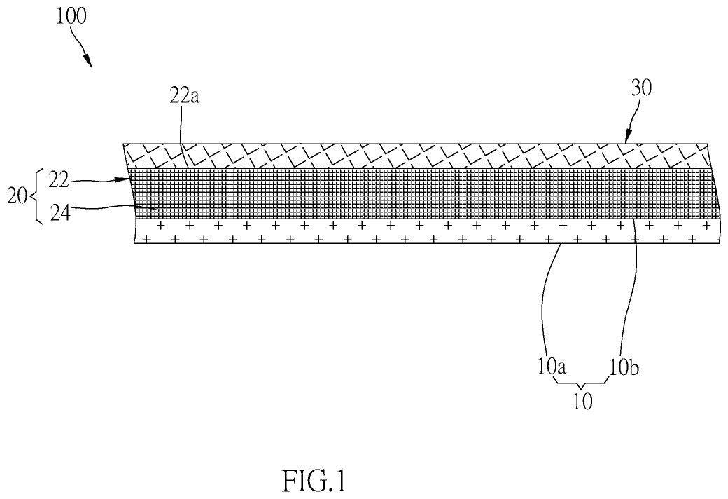

Referring to FIG. 1, illustrating a partial sectional schematic view of a skin-care product 100 according to a first embodiment of the present invention, wherein the skin-care product 100 is adapted to be attached to an inner surface of an underwear for skincare of private parts of a human body. The skin-care product 100 includes a carrier 10, a middle pad 20, and a protective film 30.

The carrier 10 is made of a waterproof and breathable material and is in a shape of a film, and has an attached surface 10a and an engaging surface 10b, wherein the attached surface 10a and the engaging surface 10b face opposite directions. The attached surface 10a is detachably attached to the inner surface of the underwear. For instance, the attached surface 10a is coated with an adhesive and is attached to a bra via the adhesive; or, the attached surface 10a is provided with a hook structure (not shown) (e.g. Velcro), thereby the attached surface 10a could be detachably hooked to fibers of a cotton underpants. The aforementioned bra and the underpants belong to the underwear of the present invention.

The middle pad 20 is disposed on the engaging surface 10b of the carrier 10 and includes a non-woven fabric base 22, wherein the non-woven fabric base 22 is impregnated with an immersion maintenance solution 24. The immersion maintenance solution 24 has a maintenance ingredient that could nourish human skin, such as, but not limited to, hyaluronic acid, vitamin liquid molecules, plant-based extracts, or a combination thereof, and such ingredients could improve a water retention and a whiteness of the skin. The non-woven fabric base 22 has a contact surface 22a, which is located at a side opposite to the engaging surface 10b and is entirely covered by the protective film 30, thereby preventing the maintenance solution 24 from leaking or evaporating or spilling before being used. In the current embodiment, the protective film 30 is a plastic film. However, in practice, the protective film 30 could be other films or membranes with waterproof characteristics.

When a user is about to perform skincare in private parts, attach the attached surface 10a of the skin-care product 100 to the inner surface of the underwear (e.g. an inner surface of the bra or an inner surface of the underpants), and remove the protective film 30 before putting on the underwear, so that the contact surface 22a of the middle pad 20 touches a skin of the human body, and the maintenance solution 24 will seep out from the contact surface 22a and nourish the skin. After a predetermined time has elapsed, the skin-care product 100 could be removed from the underwear and discarded. Or, in other practices, the attached surface of the skin-care product could be reused and the maintenance ingredients in the middle pad could be replenished, thereby achieving the purpose of reuse.

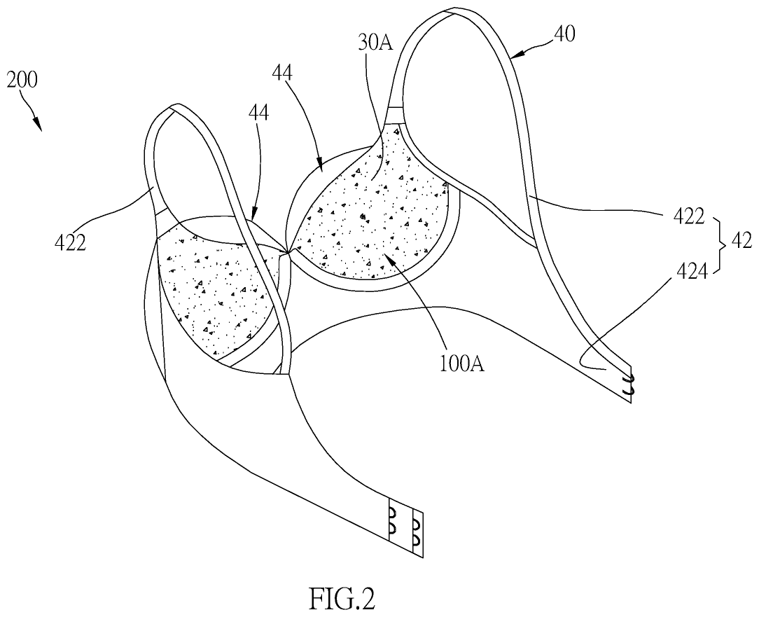

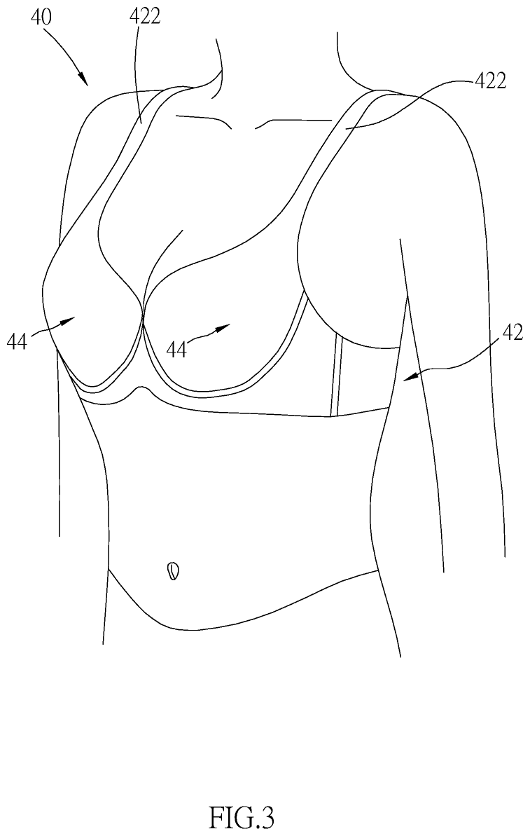

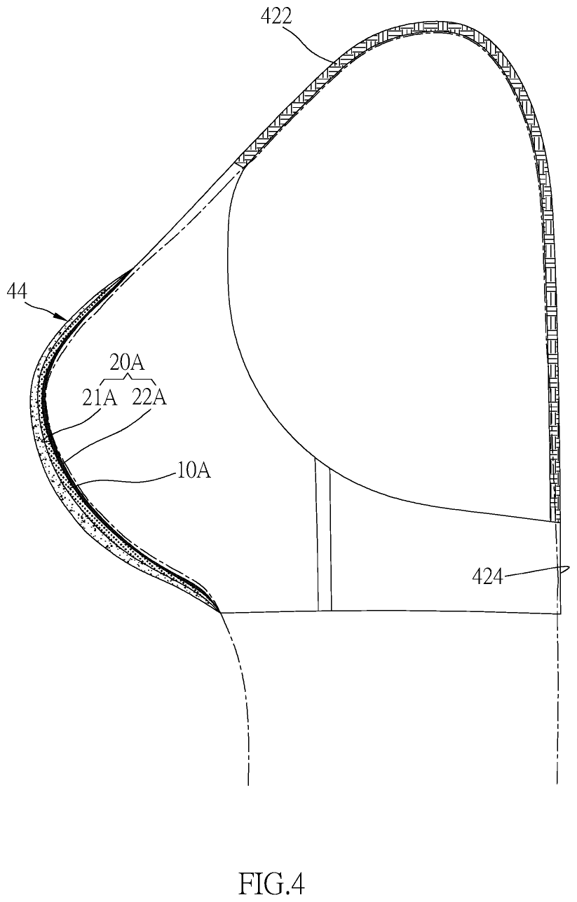

A disposable underwear with a skin-care product according to a second embodiment of the present invention is illustrated in FIG. 2 to FIG. 4, including a close-fitting fabric and a skin-care product 100A. More specifically, in the current embodiment, the disposable underwear is a disposable skin-care bra 200, wherein the close-fitting fabric is a bra fabric 40 which is made of cotton and has a body portion 42 and two cups 44. The body portion 42 has two straps 422 and a closure 424, wherein the two straps 422 are symmetrically disposed, and the two cups 44 are disposed at a front side of the body portion 42, and a part of peripheral edges of the two cups 44 are connected. In the current embodiment, the closure 424 is located at a rear side of the body portion 42.

The skin-care product 100A includes a carrier 10A, a middle pad 20A, and a protective film 30A, wherein the carrier 10A is made of a waterproof and breathable material and has the attached surface and the engaging surface as described in the first embodiment. In the current embodiment, the attached surface is fixed to an inner surface of the two cups 44. The middle pad 20A has a first surface 21A and a second surface 22A which face opposite directions, wherein the engaging surface of the carrier 10A is engaged with the first surface 21A, so that the middle pad 20A is fixed to the two cups 44. Similarly, the middle pad 20A includes the non-woven fabric base as described in the first embodiment, and the non-woven fabric base is also impregnated with the immersion maintenance solution, wherein the immersion maintenance solution has a maintenance ingredient that could nourish human skin, such as, but not limited to, hyaluronic acid, vitamin liquid molecules, plant-based extracts, or a combination thereof, and such ingredients could improve a water retention and a whiteness of the skin.

The protective film 30A is detachably and entirely attached to the second surface 22A to isolate the second surface 22A from an outside, thereby preventing the maintenance solution from leaking, evaporating, or spilling before being used. In other words, the second surface 22A of the current embodiment is equivalent to the contact surface of the first embodiment. In the current embodiment, the protective film 30A is a plastic film. However, in practice, the protective film 30 could be other films or membranes with waterproof characteristics.

When a user is about to wear the disposable skin-care bra 200, the protective film 30 is removed first, and the two straps 422 abut against shoulders of the user, and the closure 424 supports a breast of the user, so that the maintenance solution 24 will seep out from the second surface 22A and nourish the skin of the breast. When the maintenance is completed after a predetermined time, the disposable skin-care bra 200 could be taken off and discarded.

It is worth mentioning that, the skin-care product is not limited to nourish the skin of the breast, but could optionally engage the skin-care product with the inner surface of the whole bra fabric, so that the skin around the breast could be nourished as well. In addition, the straps of the bra fabric could be omitted, for the closure has provided enough fixation effect. In practice, the bra fabric could be a bustier.

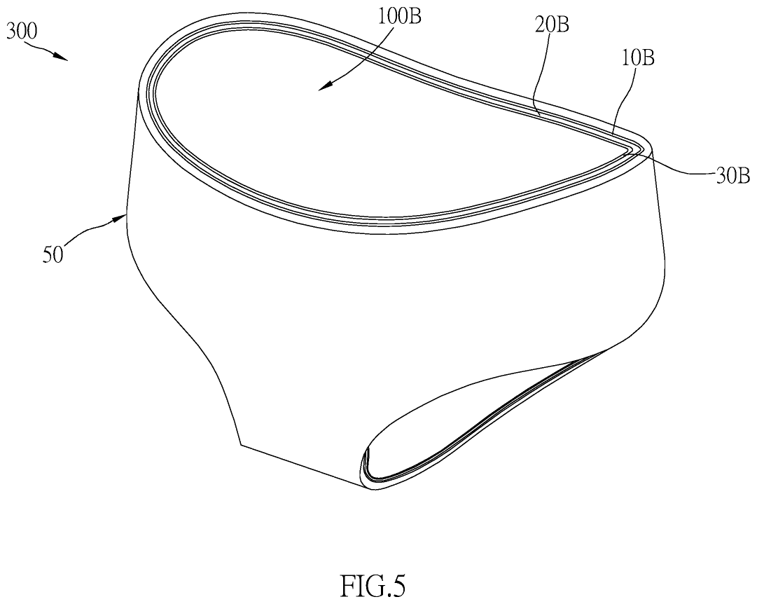

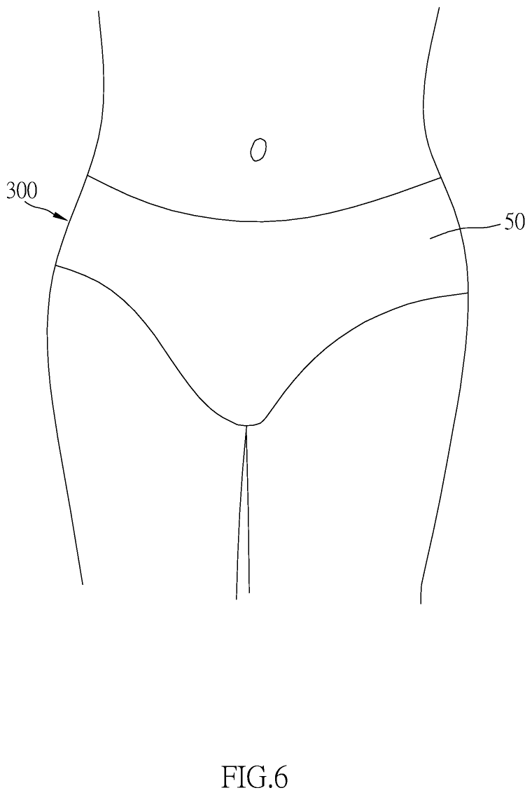

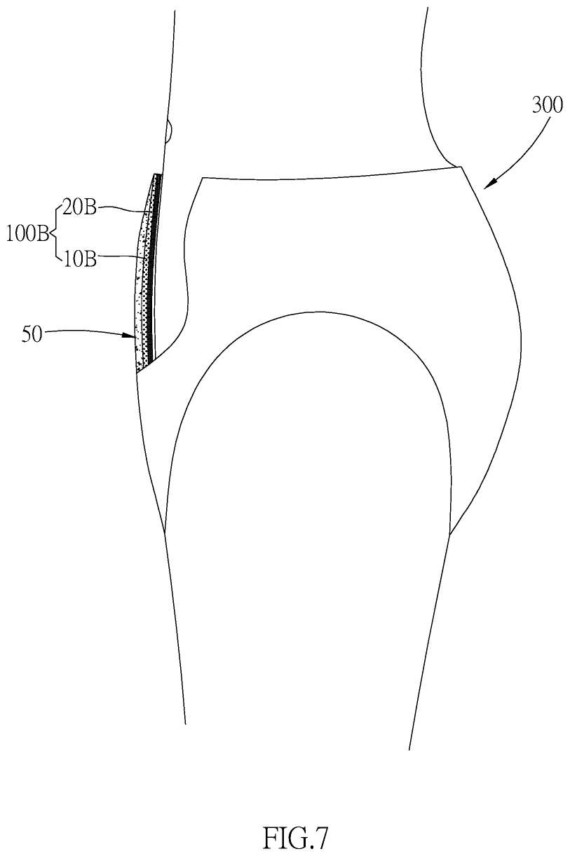

A disposable underwear with a skin-care product according to a third embodiment of the present invention is illustrated in FIG. 5 to FIG. 7, including a close-fitting fabric and a skin-care product 100B. More specifically, in the current embodiment, the disposable underwear is a disposable skin-care underpants 300, wherein the close-fitting fabric is an underpants fabric 50 such as cotton briefs covering the groin, part of the inner thigh, and the crotch of the human body.

In the current embodiment, the skin-care product includes a carrier 10B, a middle pad 20B, and a protective film 30B, wherein the carrier 10B is made of a waterproof and breathable material and has the same structures as that of the second embodiment. In the current embodiment, the attached surface is fixed to an inner surface of the underpants fabric 50, and the engaging surface is fixed to the middle pad 20B, so that the middle pad 20B is detachably engaged with the inner surface of the underpants fabric 50. The middle pad 20B of the current embodiment has the same structures as that of the second embodiment and is detachably engaged with the protective film 30B.

When a user is about to wear the disposable skin-care underpants 300, the protective film 30 is removed first, so that the maintenance solution will seep out and nourish the skin of the private part of the user. When the maintenance is completed after a predetermined time, the disposable skin-care underpants 300 could be taken off and discarded. In practice, the underpants fabric 50 is not limited to be the cotton briefs, but could be but could be boxer briefs, thongs, tangas, boyshorts, string thongs, brazilians, etc.

In other embodiments, the bra fabric of the disposable skin-care bra and the underpants fabric of the disposable skin-care underpants could be made of a waterproof material, so that the carrier of the disposable skin-care bra and the carrier of the disposable skin-care underpants could be omitted, and the middle pad could be attached to the inner surface of the bra fabric and the inner surface of the underpants fabric. Alternatively, the bra fabric and the underpants fabric could use a thicker fabric, and the fabric could be non-waterproof, and the carrier of the skin-care product is omitted, in this way, the bra fabric and the underpants fabric could gradually absorb the overflowed maintenance solution during use, while an outer surface of the bra fabric and the underpants fabric could still be kept dry.

With the aforementioned design, the skin-care product and the disposable underwear with the skin-care product of the present invention could allow the user to do other housework or other activities while maintaining the private parts, enhancing a convenience. Additionally, the carrier of the present invention is made of a waterproof material, and the underwear of the present invention is made of a waterproof material or is a thicker fabric, thereby preventing the maintenance solution from leaking or evaporating or spilling to stick to other items while using the skin-care product or the disposable underwear. Moreover, the protective film could prevent the maintenance solution from leaking, evaporating, or spilling before using the skin-care product or the disposable underwear, improving an effect of maintenance.

It must be pointed out that the embodiments described above are only some preferred embodiments of the present invention. All equivalent structures which employ the concepts disclosed in this specification and the appended claims should fall within the scope of the present invention.

What is claimed is:

1. A skin-care product, which is adapted to provide skincare of private parts of a human body and is adapted to be detachably attached to an inner surface of an underwear, wherein the skin-care product comprises a carrier, a middle pad, and a protective film, wherein the skin-care product is characterized in that:
the carrier has an attached surface and an engaging surface which face opposite directions, wherein the attached surface is adapted to be attached to the inner surface of the underwear; and
the middle pad is disposed on the engaging surface of the carrier and comprises a maintenance ingredient for nourishing a human skin and has a contact surface;
the protective film is detachably engaged with the middle pad in a way that the protective film entirely covers the contact surface of the middle pad.

2. The skin-care product as claimed in claim 1, wherein the carrier is made of a waterproof and breathable material and is in a shape of a film.

3. The skin-care product as claimed in claim 1, wherein the protective film is a film with a waterproof characteristic.

4. The skin-care product as claimed in claim 1, wherein the middle pad comprises a non-woven fabric base which is impregnated with an immersion maintenance solution.

5. The skin-care product as claimed in claim 4, wherein the maintenance solution is hyaluronic acid, vitamin liquid molecules, plant-based extracts, or a combination thereof

6. The skin-care product as claimed in claim 1, wherein the underwear is a bra or a underpants.

7. The skin-care product as claimed in claim 2, wherein the underwear is a bra or a underpants.

8. The skin-care product as claimed in claim 3, wherein the underwear is a bra or a underpants.

9. The skin-care product as claimed in claim 4, wherein the underwear is a bra or a underpants.

10. The skin-care product as claimed in claim 5, wherein the underwear is a bra or a underpants.

11. A disposable underwear with a skin-care product, wherein the disposable underwear comprises a close-fitting fabric and a skin-care product; the disposable underwear is characterized in that:
the skin-care product comprises a middle pad and a protective film, wherein the middle pad comprises a maintenance ingredient for nourishing a human skin and has a first surface and a second surface which face opposite directions; the first surface is attached to an inner surface of the close-fitting fabric; the protective film is detachably attached to the second surface and entirely covers the second surface.

12. The disposable underwear as claimed in claim 11, wherein the close-fitting fabric is waterproof

13. The disposable underwear as claimed in claim 11, wherein the skin-care product comprises a carrier made of a waterproof and breathable material; the carrier has an attached surface and an engaging surface which face opposite directions; the attached surface is attached to an inner surface of the close-fitting fabric, and the engaging surface is engaged with the first surface of the middle pad, so that the middle pad is fixed to the close-fitting fabric.

14. The disposable underwear as claimed in claim 12, wherein the middle pad comprises a non-woven fabric base which is impregnated with an immersion maintenance solution.

15. The disposable underwear as claimed in claim 14, wherein the maintenance solution is hyaluronic acid, vitamin liquid molecules, plant-based extracts, or a combination thereof

16. The disposable underwear as claimed in claim 11, wherein the protective film is a film with a waterproof characteristic.
